(19) United States
(12) Patent Application Publication
(10) Pub. No.: US 2016/0303040 A1
(43) Pub. Date: Oct. 20, 2016

(54) INJECTABLE PHARMACEUTICAL COMPOSITIONS COMPRISING PROSTACYCLIN

(71) Applicant: RIGSHOSPITALET, Kobenhavn Ø (DK)

(72) Inventors: Pär Johansson, Dösjebro (SE); Philip Hansen, Holte (DK); Jens Kindtler, Hørsholm (DK)

(73) Assignee: RIGSHOSPITALET, Kobenhavn Ø (DK)

(21) Appl. No.: 15/037,494

(22) PCT Filed: Nov. 18, 2014
(86) PCT No.: PCT/DK2014/050392

§ 371 Date: May 18, 2016

(30) Foreign Application Priority Data
Nov. 19, 2013 (DK) PA 2013 70707

(51) Int. Cl. A61K 9/00; A61K 47/18; A61K 31/5578

(52) U.S. Cl. CPC A61K 9/0019; A61K 31/5578; A61K 47/183

(57) ABSTRACT

The present invention relates to injectable pharmaceutical compositions showing improved storage stability said compositions comprising iloprost.

### FIELD OF INVENTION

The present invention relates to injectable pharmaceutical compositions showing improved storage stability. In particular, the present invention relates to compositions comprising Iloprost.

### BACKGROUND OF INVENTION

Iloprost is a synthetic analogue of prostacyclin PGI₂. Iloprost dilates systemic and pulmonary arterial vascular beds and is used as a drug to treat pulmonary arterial hypertension (PAH), scleroderma, Raynaud's phenomenon, ischemia, sepsis, multiple organ failure, acute traumatic coagulopathy, and capillary leakage. Iloprost is available for inhalation and in an intravenous form; the latter developed and marketed by Schering AG under the trade name Ilomedin®. Ilomedin® is distributed in concentrated form and is diluted prior to injection/infusion e.g. for the treatment of capillary leakage or acute traumatic coagulopathy.

Prostaglandins in general are unstable substances and iloprost has been found to be sensitive to temperature, light and acid conditions. Iloprost is an oily substance, very slightly soluble in water and at temperatures of 6 C and up, iloprost decomposes significantly, the oily substance crystallizes and gets turbid, and the amount of decomposition product increases. Thus the instability of iloprost has led to a major problem of not having ready to use formulations available.

It is common in the field of pharmaceutical formulation to use various agents for enhancing the stability of active compounds. Such agents may include excipients, chelators and the like, example of chelators include citric acid and EDTA, and compositions comprising iloprost and EDTA have been disclosed previously.

French patent application no.: FR2729823A1 concerns an iloprost composition comprising a chelator such as citric acid or EDTA. The purpose however, is to provide a liquid for sampling blood or plasma, not for providing a long-term stable ready to use formulation of iloprost. Furthermore, the concentration range given for iloprost is very wide.

Also Ruf et al (1992) Blood 80: 1238-1246, a scientific publication dedicated to the study of platelet-dependent activation of polymorphonuclear neutrophils, discloses a composition comprising iloprost and EDTA. However, the document does not disclose any effect on stability and does not disclose the preferred concentration intervals or pH of the composition of the present invention.

The thesis by Kerstin Bäcklund (May 2013, Swedish University for Agricultural Science, Uppsala, Sweden) directed towards improving platelet counting in cats by comparing types of anticoagulation in blood collection tubes discloses a composition comprising iloprost and EDTA. However no concentrations, pH or effect on long-term storage is given.

The pharmaceutical composition on the market today contains a high concentration of iloprost in order to fulfil the requirements with regard to stability. This concentrated formulation, sold under the name Ilomedin®, is diluted in isotonic sodium chloride or glucose prior to use and the diluted composition is stable less than 24 h after preparation. Under certain circumstances, such as during urgent surgical procedures, in environments where sanitary conditions are poor, or outside a regular hospital setting, a ready to use composition for injection and/or infusion would be a great advantage. This has not been possible due to the poor stability of diluted aqueous compositions containing iloprost.

Several of the disorders that are treated by administration of injection or infusion of iloprost are acute, potentially fatal and may have a very rapid onset for example sepsis, multiple organ failure, acute traumatic coagulopathy, and capillary leakage. Furthermore, several of the herein mentioned disease may occur outside of a hospital setting and thus there is a need for a ready to use composition for injection and/or infusion with long term storage capability.

Clearly, there remains a need to develop pharmaceutical compositions for injection and/or intravenous use that are prepared and stored as ready to use medicaments and which fulfil the requirements with regard to stability during periods of storage.

### SUMMARY OF INVENTION

The present invention solves this problem by providing an iloprost composition which is ready to use and stable in room temperature for at least 6 months, said compositions comprising iloprost and EDTA.

The present invention is concerned with novel ready-to-use solutions comprising iloprost having enhanced stability, which makes these solutions a superior choice to store in emergency places such as for example pharmacies, hospitals and in mobile or emergency medical aid vehicles and kits.

The present invention also relates to the use of the injectable pharmaceutical compositions in the treatment of an iloprost-requiring condition such as for example sepsis, organ failure, acute traumatic coagulopathy, and capillary leakage, such as systemic capillary leakage associated with surgery.

The preferred formulation exhibiting long term stability comprises 200 ng/ml iloprost, isotonic NaCl and 0.5 mg/ml EDTA at pH 8 in a phosphate buffered aqueous solution.

### DETAILED DESCRIPTION OF THE INVENTION

The present invention relates to injectable pharmaceutical compositions of iloprost showing improved storage stability. In particular, the present invention relates to compositions which can be stored as ready to use formulations.

The inventors of the present invention have found that an injectable pharmaceutical composition, which is stable for several months, can be obtained by the formulation comprising:
a) 150-250 ng/ml iloprost
b) A chelator selected from EDTA, EGTA, HEDTA, DTPA, NTA and/or citric acid wherein the composition has a pH 7 to 10.

It has been found that such compositions are stable for at least 6 months during storage.

The compositions of the present invention differ from commercial products, which are on the market today, because the concentration of iloprost is very high in the commercial products and the commercial product thus requires dilution prior to use.

By the term “iloprost” is herein meant the compound having the chemical formula : (E)-(3aS,4R,5R,6aS)-hexahydro-5-hydroxy-4-[(E)-(3S,4RS)-3-hydroxy-4-methyl-1-octen-6-ynyl]-Δ2(1H),Δ-pentalenevaleric acid and the IUPAC name: 5-{(E)-(1S,5S,6R,7R)-7-hydroxy-6[(E)-(3S,4RS)-3-hydroxy-4-methyl-1-octen-6-inyl]-bi-cyclo[3.3.0]octan-3-ylidene}pentanoic acid. Iloprost is sold under the tradenames Ilomedin® as a product for infusion and Ventavis® for inhalation.

Iloprost may be obtained in one of its pharmaceutical acceptable salts. In the present invention the preferred salt is trometamol.

The concentration of iloprost in the injectable compositions of the present invention lies in the range of 150 to 250 ng/ml iloprost, such as for example in the range of 175 to 225 ng/ml or 190 to 210 ng/ml iloprost. In a particularly preferred embodiment of the present invention the concentration of iloprost is 200 ng/ml.

The compositions of the present invention may further comprise a chelating agent. By the term “chelating agent” as used herein is meant a compound that is capable of forming chelating complexes or inclusion complexes with iloprost. Examples of chelating agents include EDTA and EGTA, as well as HEDTA, DTPA and NTA. The skilled person would know that citric acid may also be referred to as a chelating agent. In a preferred embodiment the chelator is EDTA.

The concentration of EDTA in the injectable compositions of the present invention lies in the range of 0.1 to 10 mg/ml such as 0.1 to 5 mg/ml such as 0.1 to 1.0 mg/ml, such as 0.2 to 0.9 mg/ml such as 0.3 to 0.8 mg/ml such as 0.3 to 0.7 mg/ml such as 0.4 to 0.6 mg/ml such as about 0.5 mg/ml. In a particular preferred embodiment of the present invention the concentration of EDTA is 0.5 mg/ml.

In an embodiment the compositions also comprise citric acid in addition to EDTA. The concentration of citric acid in the injectable compositions of the present invention lies in the range of 0.1-100 mM, such as for example in the range of 1-50 mM, such as 2.4 mM. Citric acid is in relation to the present invention particularly relevant to use at pH close to 7 such as between 7 and 8 as well as pH 8. In particular embodiments with pH close to 7, such as between 7 and 8 the iloprost composition solely comprises citric acid as chelator or antioxidant. Thus in an embodiment of the invention the injectable pharmaceutical composition comprises 150 to 250 ng/ml iloprost and citric acid and the composition has pH 7 to 8.

Sodium chloride may be added to the composition according to the present invention. In a particularly preferred embodiment the concentration of sodium chloride needed for obtaining an isotonic concentration.

The solution pH in the injectable compositions of the present invention lies in the range of pH 7 to 10, such as for example pH 7.3 to 9.5, such as a range of 7.5 to 9, or such as a range of 7.5 to 8.5. In a particularly preferred embodiment of the present invention the pH of the solution pH is 8.

Sodium phosphate maybe used as a pH buffer in the compositions according to the present invention. In a preferred embodiment of the present invention sodium phosphate is present in a concentration of 10 mM.

The preferred formulation exhibiting long term stability comprises 200 ng/ml iloprost, isotonic NaCl and 0.5 mg/ml EDTA at pH 8. Preferably, the composition is prepared in a phosphate buffered aqueous solution.

In another preferred embodiment the composition comprises 200 ng/ml iloprost in a phosphate buffered aqueous solution. Optionally, this composition also comprises isotonic sodium chloride.

The inventors of the present invention surprisingly found that isotonic glucose has a strong negative influence on the chemical stability of iloprost solutions. Accordingly, the most preferred embodiments of the present invention do not comprise isotonic glucose.

The compositions of the present invention may further comprise an antioxidant. By the term “antioxidant” as used herein is meant a material that will prevent oxidation of adrenaline. Examples of antioxidants include cysteine, thioglycerol, acetylcysteine and ascorbic acid. The skilled person will know that citric acid may also be referred to as an antioxidant. In a preferred embodiment the compositions also comprises ascorbic acid in addition to EDTA and/or citric acid.

The compositions of the present invention are formulated as injectable formulations. By the term “injectable formulation” or “injectable composition” as used herein is meant a formulation or composition, which is to be administered by injection or infusion techniques. Hence, the compositions may be administered via a parenteral route. As used herein, the term “parenteral” includes routes that bypass the alimentary tract. Hence, the term parenteral as used herein includes subcutaneous injections, intravenous, intramuscular, intrasternal, intracavernous, intrathecal injection or infusion techniques.

The injectable pharmaceutical composition is preferably sterile. It may also be desirable to include other components in the preparation, such as delivery vehicles including but not limited to aluminum salts, water-in-oil emulsions, biodegradable oil vehicles, oil-in-water emulsions, biodegradable microcapsules, and liposomes.

In a preferred embodiment the compositions of the present invention is formulated as an injectable formulation for use as an intravenous solution. Such composition may further comprise excipients approved for use in intravenous solutions as known to the person of skill in the art. In an embodiment the composition does not comprise glucose, particularly isotonic glucose.

The injectable pharmaceutical compositions of the present invention may be used in the treatment of an iloprost-requiring condition in a mammal subject in need thereof, where the mammal subject is administered an effective therapeutic amount of the composition.

By the term “iloprost-requiring condition” as used herein is meant any medical condition wherein administration of iloprost to an individual having the condition has a pharmacologically beneficial effect, such as improving at least one symptom of the medical condition or preventing the disorder from developing at all or from developing to an acute stage. In some embodiments the iloprost-requiring condition is pulmonary arterial hypertension (PAH), scleroderma, Raynaud's phenomenon, ischemia, sepsis, organ failure, acute traumatic coagulopathy, and capillary leakage. In particular the iloprost-requiring condition that may be treated or prevented is selected from the group consisting of organ failure, such as organ failure due to severe infection or sepsis, sepsis, acute traumatic coagulopathy, and capillary leakage such as systemic capillary leakage associated with surgery.

The mammal subjects to be treated are preferably human beings. However, other subjects, such as for example dog, cat, horse, cow, goat and sheep may also be treated by the composition of the present invention.

Pharmaceutical compositions of the present invention comprise an effective amount of adrenaline. The adrenaline may be dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains iloprost and/or EDTA and optionally other pharmaceutical acceptable excipients will be known to those of skill in the art in light of the present disclosure, as exemplified by Remington's Pharmaceutical Sciences, 18th Ed. Mack Printing Company, 1990, incorporated herein by reference.

The injectable pharmaceutical compositions show superior storage stability. Moreover, since the compositions are formulated as ready-to-use formulations, the compositions are particular suitable for storage in emergency places such as pharmacies, hospitals and/or homes, in mobile or emergency medical aid kits, for travelers (especially to remote areas), for medical facilities lacking reliable refrigerated storage or hygienic conditions for sterile reconstitution of an injectable drug, and other contexts where stable, long-term storage of a stable pharmaceutical solution at ambient temperature may offer convenience, safety and/or cost-savings. Optionally preservatives may be added to the formulations. In preferred embodiment there are no preservatives in the composition according to the present invention as preservatives are not needed in single dose containers.

The composition according to the present invention may be stored in an infusion bag or bottle e.g. for infusion pump. Furthermore, the container for storage is may be protected against daylight and/or oxygen. The protection against oxidation may include argon or nitrogen in head space. Also, the product may be subject to terminal sterilization.

### EXAMPLES

Analytical protocol for quantification of iloprost in new formulations of the compound. of the project

1. Set-up and validation of analytical LC-MS analysis for quantification of iloprost in an aqueous formulation. The method provided for a quantitative detection of iloprost in the concentration range from 10 to 1000 ng/ml.

2. Analysis of samples after 1, 3, and 6 month storage. Each run of 30±10 samples required re-validation of the method, including preparation of new standard curve and analysis of QC samples. MS-chromatograms were scanned for the major known degradation products of iloprost.

### Instrumentation

LC-MS analysis was conducted on a LC-MS manufactured by Agilent Technologies and includes a 1200 Series LC and a 6140 Quadropole MS equipped with a 6140 Multimode detector running ChemStation B.04.01 software.

### Stock Solutions and Standard Solutions

A: A concentration iloprost equal to 20 μg/ml (was used for the optimization of the HPLC procedure, UV 210 nm)

B: 0.9% sodium chloride in milliQ water (0.9 g NaCl in 100 ml water)

C: 100 μl of A and 900 μl of B: iloprost concentration equal to 2 μg/ml

A 10 days short term stability study was conducted on the standards The test solutions were prepared 11 May 2012 and analysed 11 May 2012, n=2, 15 May 2012, and 21 May 2012 and finally 21 May 2012. Between the days of analysis the samples were stored at 5° C. No indication on major instability of iloprost after dilution with an isotonic sodium chloride solution was observed.

Linear response curves for iloprost were obtained in the concentration range from 30 to 500 ng/ml. The method covered analysis of iloprost in samples with a LOQ equal to 30 ng/ml, with an accuracy CV % <10. Accuracy assessment (non-regulated) met regulated acceptance criteria. The analytical method was, therefore, deemed fit for purpose i.e. fit to determine concentrations of iloprost in isotonic sodium chloride solutions.

### Stability Studies

12 different iloprost formulations and 1 Ilomedin® reference were manufactured. They were based on the compositions of Table 1 all comprising 200 ng/ml Iloprost and water for injection to make 1 ml and all but IL-01-37 also comprised Na-phosphate (pH buffer) of 10 mM. All diluted samples contain traces from Ilomedin® excipients: Trometamol 2.35 μg/ml-NaCl 90 μg/ml-Ethanol 16 μg/ml. In the Ilomedin® reference (IL-01-37) Ilomedin® concentrate was diluted with 0.9% NaCl solution (as recommended by the product insert instruction)

TABLE 1
Test compositions

| | | | | NaCl | Glucose | | |
|---|---|---|---|---|---|---|---|
| Code | NaCl | Glucose | Ref. | EDTA | EDTA | EDTA | pH |
| IL-01-01 | 9 mg/ml | | | | | | 8 |
| IL-01-02 | | 50 mg/ml | | | | | 8 |
| IL-01-03 | | | No | | | | 8 |
| | | | excipient | | | | |
| IL-01-04 | | | | 9 & 0.5 mg/ml | | | 8 |
| IL-01-05 | | | | | 50 & 0.5 mg/ml | | 8 |
| IL-01-06 | | | | | | 0.5 mg/ml | 8 |
| IL-01-07 | 9 mg/ml | | | | | | 9 |
| IL-01-08 | | 50 mg/ml | | | | | 9 |
| IL-01-09 | | | No | | | | 9 |
| | | | excipient | | | | |
| IL-01-10 | | | | 9 & 0.5 mg/ml | | | 9 |
| IL-01-11 | | | | | 50 & 0.5 mg/ml | | 9 |
| IL-01-12 | | | | | | 0.5 mg/ml | 9 |
| IL-01-37 | 9 mg/ml | | | | | | 7 |

TABLE 2
Stability matrix:

| | Time | | | |
|---|---|---|---|---|
| | 1 month | 3 months | 6 months | Condition |
| 40 C. | X | X | | Day light |
| 25 C. | X | X | X | Day light |
| 5 C. | X | X | X | Darkness |

### Results

TABLE 3.1
Stored at 5 C. in darkness for 1, 3 or 6 months

| | | | 5 C. | | |
|---|---|---|---|---|---|
| | | Mth | 1 | 3 | 6 |
| pH 8 | NaCl | IL-01-01 | 221 | 207 | 215 |
| | Glucose | IL-01-02 | 190 | 204 | 236 |
| | | IL-01-03 | 212 | 210 | 211 |
| | NaCl/EDTA | IL-01-04 | 194 | 210 | 223 |
| | Gluc/EDTA | IL-01-05 | 191 | 202 | 200 |
| | EDTA | IL-01-06 | 219 | 212 | 205 |
| pH 9 | NaCl | IL-01-07 | 233 | 203 | 211 |
| | Glucose | IL-01-08 | 204 | 197 | 208 |
| | | IL-01-09 | 263 | 215 | 213 |
| | NaCl/EDTA | IL-01-10 | 209 | 198 | 218 |
| | Gluc/EDTA | IL-01-11 | 213 | 201 | 195 |
| | EDTA | IL-01-12 | 233 | 209 | 200 |
| | Ilomedin ® | IL-01-37 | 202 | 183 | 204 |

TABLE 3.2
Stored at 25 C. in day light for 1, 3 or 6 months

| | | | 25 C. | | |
|---|---|---|---|---|---|
| | | Mth | 1 | 3 | 6 |
| pH 8 | NaCl | IL-01-01 | 217 | 206 | 206 |
| | Glucose | IL-01-02 | 195 | 192 | 193 |
| | | IL-01-03 | 221 | 214 | 207 |
| | NaCl/EDTA | IL-01-04 | 206 | 210 | 224 |
| | Gluc/EDTA | IL-01-05 | 205 | 200 | 193 |
| | EDTA | IL-01-06 | 217 | 216 | 209 |
| pH 9 | NaCl | IL-01-07 | 216 | 198 | 203 |
| | Glucose | IL-01-08 | 182 | 187 | 190 |
| | | IL-01-09 | 228 | 210 | 206 |
| | NaCl/EDTA | IL-01-10 | 196 | 200 | 210 |
| | Gluc/EDTA | IL-01-11 | 204 | 196 | 187 |
| | EDTA | IL-01-12 | 212 | 210 | 199 |
| | Ilomedin ® | IL-01-37 | 209 | 180 | 192 |

TABLE 3.2
Stored at 40 C. in day light for 1 or 3 months

| | | | 40 C. | | |
|---|---|---|---|---|---|
| | | Mth | 1 | 3 | 6 |
| pH 8 | NaCl | IL-01-01 | 230 | 205 | |
| | Glucose | IL-01-02 | 192 | 189 | |
| | | IL-01-03 | 237 | 218 | |
| | NaCl/EDTA | IL-01-04 | | 219 | |
| | Gluc/EDTA | IL-01-05 | 208 | 188 | |
| | EDTA | IL-01-06 | 221 | 230 | |
| pH 9 | NaCl | IL-01-07 | 219 | 200 | |
| | Glucose | IL-01-08 | 182 | 157 | |
| | | IL-01-09 | 235 | 218 | |
| | NaCl/EDTA | IL-01-10 | 201 | 204 | |
| | Gluc/EDTA | IL-01-11 | 198 | 174 | |
| | EDTA | IL-01-12 | 219 | 216 | |
| | Ilomedin ® | IL-01-37 | 219 | 175 | |

The results show that:
ready to use Iloprost 200 ng/ml solutions have a surprisingly good long-term stability
the best results are obtained with isotonic sodium chloride and EDTA 0.5 mg/ml, but also in a solution without any of the tested excipients.
pH 8 seems to be slightly better than pH 9.

All in all it may be concluded that the data indicate that iloprost 200 ng/ml at pH 7-9 can be stabilized with sodium chloride and EDTA as stabilizers.

1. An injectable pharmaceutical composition, comprising:
a. 150 to 250 ng/ml iloprost, and
b. a chelator selected from EDTA and/or citric acid,
wherein the composition has pH 7 to 10.

2. The composition according to claim 1, wherein the concentration of iloprost is 200 ng/mL.

3. The composition according to claim 1 or 2, wherein the solution pH lies in the range of 7-10, such as 7.3 to 9, such as 8 to 9, such as 7.5 to 8.5, such as 7.7 to 8.3, preferably 8.

4. The composition according to any of the preceding claims, comprising 0.5 mg/ml EDTA.

5. The composition according to any of the preceding claims further comprising citric acid, optionally in a concentration of 2.4 mg/ml, and the solution pH is 7 to 8.

6. The composition according to any of the preceding claims, comprising 200 ng/ml iloprost, 0.5 mg/ml EDTA and where the solution pH is 8.

7. The composition according to any of the preceding claims, wherein the composition further comprises isotonic sodium chloride.

8. The composition according to any of the preceding claims, wherein the composition further comprises a phosphate buffer.

9. The composition according to any of the preceding claims, wherein the composition further comprises an antioxidant, such as ascorbic acid.

10. The composition according to any of the preceding claims, wherein the composition is formulated as an injectable formulation for use as an intravenous solution.

11. The composition according to any of the preceding claims wherein the chelator is selected from EDTA, EGTA, HEDTA, DTPA and/or NTA.

12. A method of treatment or prevention of an iloprost-requiring condition in a mammal subject in need thereof, said method comprising administering an effective therapeutic amount of the composition according to any of claims 1 to 11 to the mammal subject.

13. The method according to claim 12 wherein the prostacyclin/iloprost requiring condition is selected from the group consisting of acute traumatic coagulopathy, organ failure, such as organ failure due to severe infection and/or sepsis, and/or capillary leakage, such as systemic capillary leakage associated with surgery.

14. The method according to claim 12 or 13, wherein the mammal subject is a human being.

15. The method according to any of claims 12 to 14, wherein the composition comprises 150 to 250 ng/ml iloprost, preferably 200 ng/ml iloprost, and EDTA, preferably 0.5 mg/ml EDTA, and/or citric acid, preferably 2.4 mg/ml citric acid, isotonic sodium chloride, a phosphate buffer and pH 7 to 10.
